(19) United States
(12) Patent Application Publication
(10) Pub. No.: US 2004/0031674 A1
(43) Pub. Date: Feb. 19, 2004

(54) Workup of (meth)acrylic acid and (meth)acrylic esters

(75) Inventor: Jurgen Schroder, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Akiengesellschaft, Ludwigshafen (DE)

(21) Appl. No.: 10/459,612

(22) Filed: Jun. 12, 2003

(30) Foreign Application Priority Data
Aug. 15, 2002 (DE) 10238142.9

(51) Int. Cl. B01D 3/42; B01D 3/00; B01D 3/34; C01D 1/00; C07B 31/00; C07C 51/42

(52) U.S. Cl. 203/2; 203/8; 203/49; 203/DIG. 21; 562/600

(57) ABSTRACT

A process for working up mixtures containing (meth)acrylic acid and/or (meth)acrylic ester in a column for distilling, rectifying and/or fractionally condensing in the presence of at least one polymerization inhibitor and an oxygen-containing gas, wherein the partial oxygen pressure p(O₂) in the gas phase of the entire column is from 2 to 5 hPa.

The present invention relates to a process for reducing the polymerization in the distillative workup of (meth)acrylic acid and (meth)acrylic esters

Polymers prepared from (meth)acrylic acid are used, for example, as water-absorbing resins in superabsorbents. (Meth)acrylic acid is also a precursor for (meth)acrylic esters. The polymers and copolymers prepared on the basis of (meth)acrylic esters in the form of polymer dispersions are of great economic significance, for example as adhesives, paints, or textile, leather and paper assistants.

It is known that polymerizable compounds such as (meth)acrylic acid and (meth)acrylic esters can be easily polymerized, for instance by heat or reaction of light or peroxides. However, since polymerization has to be reduced or prevented for safety and economic reasons when preparing, working up and/or storing, there is a constant need for novel, simple and effective methods for reducing the polymerization.

It is well-known prior art that the polymerization of (meth)acrylic acid and (meth)acrylic esters may be suppressed by using polymerization inhibitors, frequently in combination with oxygen-containing gases.

In the case of distillation columns, these oxygen-containing gases are generally metered into the bottom. EP-A 1 035 102 describes a process for purifying (meth)acrylic acid and (meth)acrylic esters in which an oxygen-containing gas is metered into the circuit of the evaporator.

These processes in which oxygen-containing gases are metered into the bottom have the disadvantage that they do not effectively prevent the polymerization in the upper section of the distillation columns, in which (meth)acrylic acid and (meth)acrylic ester are present in high purity.

Journal of Polymer Science, Polymer Chemistry Edition, volume 23, 1985, pages 1505 to 1515 states that hydroquinone monomethyl ether is effective as a polymerization inhibitor only in the presence of oxygen. In contrast, although phenothiazine is also effective in the absence of oxygen, it is gradually destroyed by the action of oxygen. In other words, when phenothiazine is used as a polymerization inhibitor, the presence of oxygen is detrimental.

In Plant/Operations Progress, volume 10, 1991, pages 171 to 183, the influence of oxygen on the stabilization of methacrylic acid is described. Oxygen traces increase the effectiveness of the polymerization inhibitors investigated. However, phenothiazine is distinctly less effective under air than under nitrogen.

Journal of Polymer Science, Polymer Chemistry Edition, volume 30, 1992, pages 569 to 576 states that phenothiazine is effective in the absence of oxygen. In the presence of phenothiazine, no oxygen is consumed by inhibition reactions.

Farbe+Lack, volume 100, 1994, pages 604 to 609 subdivides polymerization inhibitors into aerobic and anaerobic stabilizers. Aerobic stabilizers are only effective in the presence of oxygen, and examples of such aerobic stabilizers include phenolic polymerization inhibitors, such as hydroquinone monomethyl ether. In contrast, anaerobic polymerization inhibitors, for example phenothiazine, do not require any oxygen, but rather are consumed by oxygen in non-polymerization-inhibiting secondary reactions.

Chemical Engineering Technology, volume 21, 1998, pages 829 to 837 describes the influence of oxygen on polymerization inhibition. Above 70° C., phenothiazine reacts more rapidly with radicals than with oxygen, and the oxygen consumption falls markedly.

Despite the comprehensive investigations on the stabilization of acrylic acid, it is not possible to prevent polymer fouling in distillative process steps using acrylic acid. In contrast to, for example, styrene, the polymers formed in acrylic acid are insoluble in acrylic acid, and the resulting polymer fouling therefore has to be removed regularly, which leads to costs and production outages.

JP 07053449 [Derwent Abstract No. 95-128282/17] describes the polymerization inhibition of (meth)acrylic acid and (meth)acrylates by a combination of phenothiazine with hydroquinone, hydroquinone monomethyl ether, p-benzoquinone or copper dibutyldithiocarbamate in the presence of 0.01-5% by volume of oxygen based on the total amount of (meth)acrylic acid and (meth)acrylates, and synergistic action is exhibited by phenothiazine and hydroquinone, and also to a limited extent hydroquinone monomethyl ether, in the presence of oxygen. The pressure, for example in a distillation, is set at 100-500 mmHg (approx. 130-660 hPa) which corresponds to a partial oxygen pressure P(O₂) of from 0.012 to 33 hPa. This document also discloses that phenothiazine, hydroquinone, hydroquinone monomethyl ether and para-benzoquinone exhibit a better effectiveness in the stabilization of acrylic acid in the presence of oxygen than under nitrogen, and only copper dibutyldithiocarbamate exhibits a lesser effectiveness in the presence of oxygen. While the phenothiazine/hydroquinone combination and also to a limited extent phenothiazine/hydroquinone monomethyl ether in the presence of oxygen act synergistically, the phenothiazine/para-benzoquinone combination and the phenothiazine/copper dibutyldithiocarbamate combination diminishes in the presence of oxygen.

A disadvantage of this process is that the inhibitors are not totally effective at low partial oxygen pressure. EP-A1 1 134 212 describes the preparation of hydroxyalkyl (meth)acrylates by reacting (meth)acrylic acid with an alkylene oxide in the presence of 0.1-14% by volume of oxygen, in order to prevent the formation of explosive alkylene oxide/oxygen mixtures and, at the same time, to ensure the presence of oxygen for activating the inhibitors used. The pressure is specified as 0.1-1 MPa, and a lower pressure is described as disadvantageous only because the alkylene oxide cannot be maintained in the liquid state. This corresponds to a partial oxygen pressure P(O₂) of from 1 to 140 hPa.

The process described does not recognize that critical limits of the partial oxygen pressure have to be attained, in order to confer good effectiveness on inhibitors.

When the oxygen content is high, the offgases of the preparative process for (meth)acrylic acid or (meth)acrylic esters, with or without the presence of an alkylene oxide, form an explosive mixture which has to be avoided at all costs for safety reasons. For this reason, an inert gas is added, but this increases the amount of gas and thus burdens vacuum units with an increased gas ballast.

For example, the industrial distillation of acrylic acid at 100 hPa and a partial oxygen pressure of more than 5 hPa using air as the oxygen-containing gas and a reflux ratio of more than 2 results in more than 200 m³ of offgas per metric ton of acrylic acid removed, which greatly burdens the industrially used vacuum unit.

It is an object of the present invention to provide a process for preparing (meth)acrylic acid and (meth)acrylic esters which allows the polymerization during the distillation to be effectively reduced by simple means while the gas ballast at the same time remains within an optimum range.

We have found that this object is achieved by a process for working up mixtures comprising (meth)acrylic acid and/or (meth)acrylic ester in a column for distilling, rectifying and/or fractionally condensing in the presence of at least one polymerization inhibitor and an oxygen-containing gas, wherein the partial oxygen pressure P(O₂) in the gas phase of the entire column is from 2 to 5 hPa.

It has been found that, surprisingly, the effectiveness of anaerobic polymerization inhibitors may be distinctly increased by oxygen and that even when relatively high oxygen concentrations are used, there is no negative effect on the stabilizer action.

The workup is generally effected in a column by a distillative or rectificative route or by a fractional condensation.

Examples of mixtures which can be used in accordance with the invention are those which comprise at least 5% by weight, preferably at least 10% by weight, more preferably at least 25% by weight, even more preferably at least 75% by weight and in particular at least 90% by weight, of acrylic acid or methacrylic acid, referred to hereinbelow as (meth)acrylic acid, or (meth)acrylic esters. Examples of (meth)acrylic esters include methyl, ethyl, n-propyl, iso-propyl, n-butyl, sec-butyl, iso-butyl, tert-butyl, pentyl, hexyl, octyl, 2-ethylhexyl and dodecyl (meth)acrylate.

Preferred monomers are methacrylic acid and acrylic acid, and particular preference is given to acrylic acid.

The mixture containing (meth)acrylic acid and/or (meth)acrylic esters is generally fed to the column in gaseous form, i.e. as a hot gas mixture, or in liquid form or in a mixed gaseous/liquid form.

Useful hot gas mixtures are those gas mixtures which occur as reaction gas mixtures in the catalytic gas phase oxidation of C₃-alkanes, -alkenes, -alkanols and/or -alkanals and/or precursors thereof to give acrylic acid by known processes. Propene, propane or acrolein are particularly advantageously used. However, other useful starting compounds are those from which the actual C₃ starting compound is formed only during the gas phase oxidation as an intermediate. Acrylic acid may also be prepared directly from propane. When propane is used as a starting material, this may be reacted by known catalytic oxydehydrogenation, homogeneous oxydehydrogenation or catalytic dehydrogenation processes to give a propene/propane mixture. Other useful propene/propane mixtures are refinery propane (approx. 70% of propene and 30% of propane) or cracker propene (approx. 95% of propene and 5% of propane). When a propene/propane mixture is used for preparing acrylic acid, propane is effective as a diluent gas and/or reactant. When acrylic acid is prepared, the starting gas is generally diluted with gases which are inert under the chosen reaction conditions, such as nitrogen (N₂), CO₂, saturated C₁-C₆-hydrocarbons and/or steam, and passed in a mixture with oxygen (O₂) or an oxygen-containing gas at elevated temperatures (customarily from 200 to 450° C.) and also optionally elevated pressure over transition metal (e.g. Mo and V, or Mo, W, Bi and Fe-containing) mixed oxide catalysts and oxidatively converted to acrylic acid. These reactions can be carried out in a plurality of stages or a single stage.

In addition to the desired acid, the resulting reaction gas mixture contains secondary components such as unconverted acrolein and/or propene, steam, carbon monoxide, carbon dioxide, nitrogen, oxygen, acetic acid, propionic acid, formaldehyde, further aldehydes and maleic acid or maleic anhydride. Customarily, the reaction gas mixture, based in each case on the entire reaction gas mixture, contains from 1 to 30% by weight of acrylic acid, from 0.01 to 1% by weight of propene and from 0.05 to 1% by weight of acrolein, from 0.05 to 10% by weight of oxygen, from 0.01 to 3% by weight of acetic acid, from 0.01 to 2% by weight of propionic acid, from 0.05 to 1% by weight of formaldehyde, from 0.05 to 2% by weight of other aldehydes, from 0.01 to 0.5% by weight of maleic acid and maleic anhydride, and also small amounts of acetone and a remainder of inert diluent gases. The inert diluent gases present are in particular saturated C₁-C₆ hydrocarbons, such as methane and/or propane, and in addition steam, carbon oxides and nitrogen.

Methacrylic acid may be prepared in a similar manner from C₄-alkanes, -alkenes, -alkanols and/or -alkanals and/or precursors thereof, for example from tert-butanol, isobutene, isobutane, isobutyraldehyde, methacrolein, isobutyric acid or methyl tert-butyl ether.

In addition to (meth)acrylic acid, a (meth)acrylic acid-containing mixture may also contain a solvent.

The solvent may also be used in a preceding absorption and/or extraction and includes those substances usable for this purpose and known to those skilled in the art, for example water, methyl acrylate, ethyl acrylate, butyl acrylate, ethyl acetate, butyl acetate, biphenyl, diphenyl ether, dimethyl ortho-phthalate, diethyl ortho-phthalate, dibutyl ortho-phthalate and mixtures thereof.

Preference is given to adding water or a mixture of diphenyl ether and biphenyl, preferably in a weight ratio of from 10:90 to 90:10, or a mixture to which from 0.1 to 25% by weight (based on the total amount of biphenyl and diphenyl ether) of at least one ortho-phthalate ester, e.g. dimethyl ortho-phthalate, diethyl ortho-phthalate or dibutyl ortho-phthalate, has additionally been added.

In the case of the rectificative separation of liquids or of the absorption of gases containing (meth)acrylic compounds having a flash point (determined to DIN EN 57) of below 50° C., very particular preference is given to a molecular oxygen content of the oxygen-containing gas used of from 4 to 10%.

When a (meth)acrylic ester-containing mixture is conducted into the column, this may, in addition to (meth)acrylic ester, also contain (meth)acrylic acid, water, a solvent forming an azeotrope with water, for example n-pentane, n-hexane, n-heptane, cyclohexane, methylcyclohexane, benzene, toluene or xylene, an esterification catalyst, for example sulfuric acid, phosphoric acid, alkanesulfonic acids (e.g. methanesulfonic acid, trifluoromethanesulfonic acid) and arylsulfonic acids (e.g. benzene-, p-toluene- or dodecylbenzenesulfonic acid), a transesterification catalyst, for example titanium tetraalkoxide, and natural polymers and oligomers, for example Michael addition products which are formed by adding alcohols or (meth)acrylic acid to the double bond of (meth)acrylic compounds, for example alkoxypropionic acids or acryloxypropionic acids, and also their esters.

The column into which the (meth)acrylic acid- or (meth)acrylic ester-containing mixture is conducted may be a distillation, rectification or reaction column or a column for fractional condensation.

The mixture may optionally be directly or indirectly cooled or heated beforehand, for example using a quench, for example spray coolers, Venturi scrubbers, bubble columns or other apparatus having sprayed surfaces, or tube bundle or plate heat exchangers.

The column is one of a design known per se having separating internals installed and at least one means of condensation in the top region.

Useful column internals are in principle any common internals, in particular trays, structured packings and/or random packings. Among the trays, preference is given to bubble-cap trays, sieve trays, valve trays, Thormann trays and/or dual-flow trays, and among the dumped packings, preference is given to those comprising rings, spirals, saddles, Raschig, Intos or Pall rings, barrels or Intalox saddles, Top-Pak, etc. or braids. It will be appreciated that it is also possible to combine separating internals.

Typically, the total number of theoretical plates in the column is from 5 to 100, preferably from 10 to 80, more preferably from 20 to 80 and most preferably from 50 to 80.

In the case of a column for fractional condensation, the working pressure in the column is generally from 0.5 to 5 bar (absolute), frequently from 0.5 to 3 bar (absolute) and in many cases from 0.5 to 2 bar (absolute), and in the case of a rectification column, the pressure is generally from 10 mbar to atmospheric pressure, preferably from 20 mbar to atmospheric pressure, more preferably from 20 to 800 mbar, even more preferably from 20 to 500 mbar, in particular from 30-300 mbar and especially from 50 to 200 mbar.

The feed of the mixture is not decisive for the invention, and it is generally effected in the lower half of the column, preferably in the lower third.

The reflux at which the column is operated is likewise not relevant for the invention. The reflux may be, for example, from 100:1 to 1:100, preferably from 50:1 to 1:50, more preferably from 20:1 to 1:20 and most preferably from 10:1 to 1:10, but may also be zero (no reflux).

The removal point of the product to be purified in the column is not decisive for the invention. In general, a column has at least two removal means for product streams, customarily one at the top and one at the bottom, and also optionally one or more sidestream takeoffs. For example, the product may be removed via the top or ia at least one sidestream takeoff. In the latter case, the removal may be in liquid or gaseous form. Preference is given to removing via a sidestream takeoff.

The oxygen-containing gas used is preferably air or a mixture of air and a gas which is inert under the reaction conditions. The inert gas used may be nitrogen, helium, argon, carbon monoxide, carbon dioxide, steam, lower hydrocarbons or their mixtures. The oxygen content of the oxygen-containing gas may be, for example, up to 21% by volume, preferably from 1 to 21% by volume, more referably from 5 to 21% by volume and most preferably from 10 to 20% by volume. It will be appreciated that it is also possible, if desired, to use higher oxygen contents, for example up to 50% by volume.

The amounts of oxygen-containing gas metered in is not limited in accordance with the invention. It is advantageously from 0.004 to 2.5 times the mixture conducted into the column (based in each case on the weight), preferably from 0.004 to 1 times, more preferably from 0.08 to 0.5 times and most preferably from 0.1 to 0.5 times. It will be appreciated that greater or lesser amounts are also conceivable.

It is decisive for the invention that the partial oxygen pressure P(O₂) in the gas phase of the entire column is from 2 to 5 hPa, preferably from 2 to 4.5 hPa, more preferably from 2 to 4 hPa and most preferably from 2.5 to 4 hPa.

Generally, the liquid hourly space velocity of a column operated in accordance with the invention is generally 0.07-180 metric tons/m²×h, preferably 0.7-10 metric tons/m²×h, more preferably 2-10 metric tons/m²×h, even more preferably 3.5-6 metric tons/m²×h and in particular 5-6 metric tons/m²×h.

In order to ensure the partial oxygen pressure P(O₂) which is advantageous in accordance with the invention over the entire column, it may be advantageous to feed in the oxygen-containing gas not only in the circulation evaporator, but additionally at at least one point in the column.

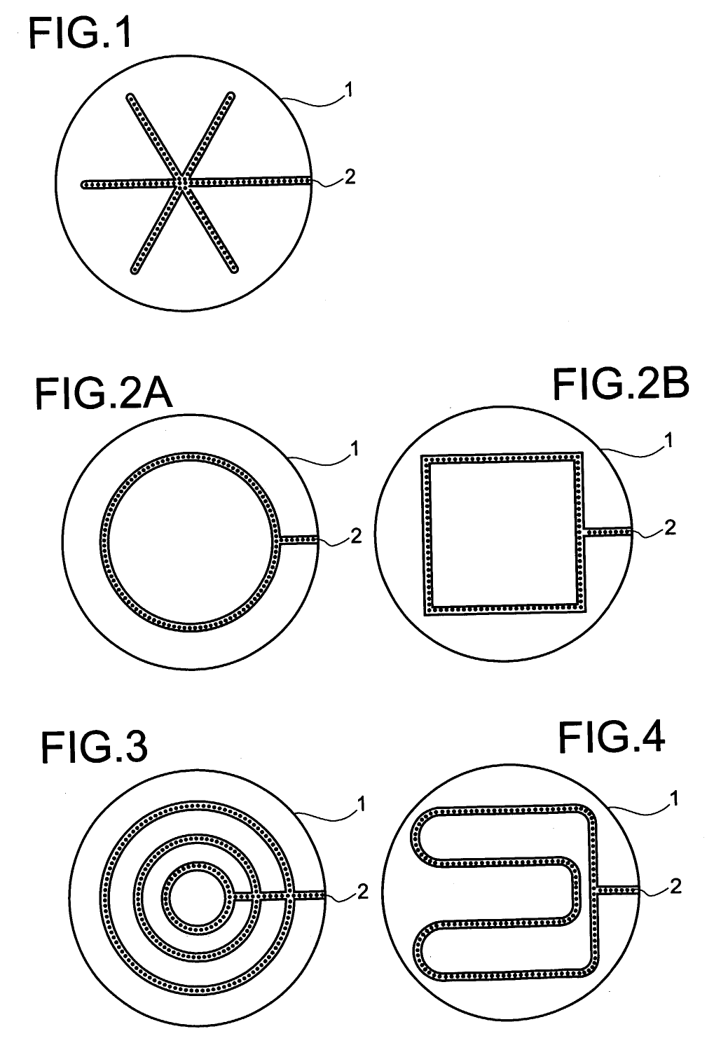

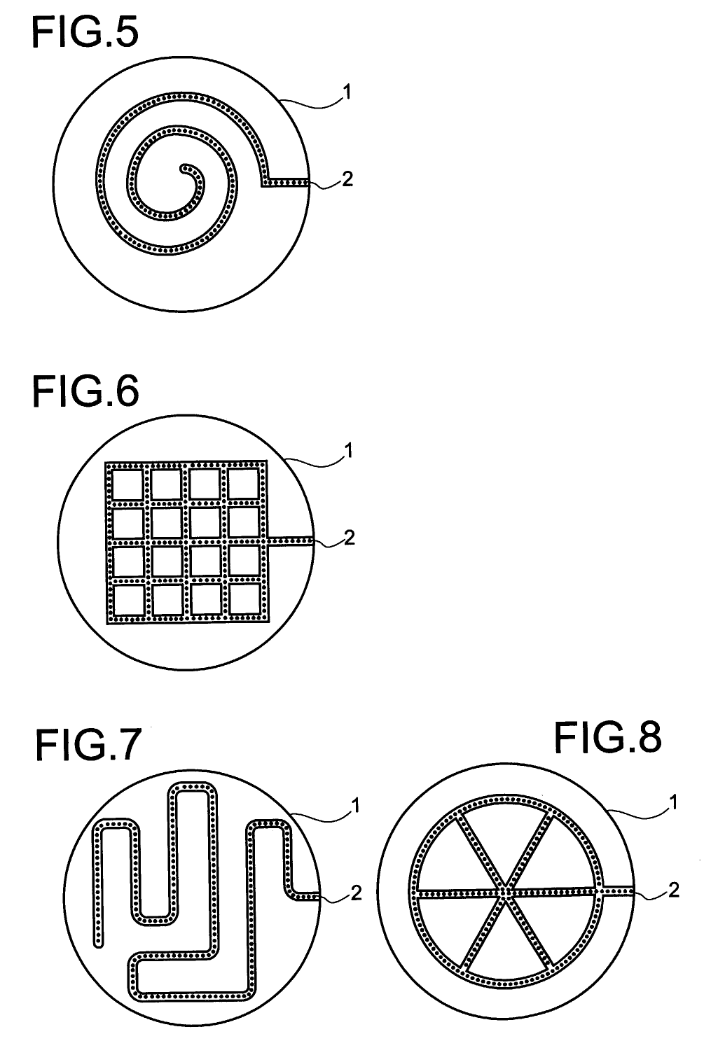

The oxygen-containing gas may be fed in via any desired devices, for example tubes, slits, nozzles or valves mounted in the column wall in the center or on the sides, preferably via those metering devices which allow a uniform distribution of the oxygen-containing gas over the surface of the separating internals (1 in the figures). The devices are preferably lines, e.g. tubes or hoses, which diverge in a star shape from the center of the surface (FIG. 1) and whose walls have openings through which the oxygen-containing gas can flow out, one (FIG. 2a) or more (FIG. 3) lines bent into concentric circles or of another regular shape, e.g. oval or rectangular or hexagonal (FIG. 2b), lines laid over the surface (1) in a coil shape (FIG. 4), lines bent in a spiral shape (FIG. 5), or lines arranged in a grid shape (FIG. 6) or irregularly, as in FIG. 7, for example, or combinations thereof (for example FIG. 8), in each case likewise having appropriate orifices. The lines may be, for example, charged with oxygen-containing gas via at least one external feed (2 in the figures). Particular preference is given to lines bent into a circle.

The material from which the metering devices are manufactured is not decisive for the invention, although it should be corrosion-stable toward the mixture to be separated in the column under the conditions in the column. They are preferably manufactured from stainless steel or copper or from copper-plated material, although plastics which are stable under the conditions in the column, e.g. Teflon® or Kevlar® are also conceivable.

The orifices in the devices may be, for example/holes, slits, valves or nozzles, preferably holes. The orifices may be distributed at any desired point over the metering devices, for example distributed on the upper and/or lower side and/or on the walls and/or randomly over the surface of the metering devices.

The number of metering devices in the column is dependent upon the type and number of separating internals. At the minimum, at least one device is installed in the upper section of the column. As the upper limit, there should sensibly be one metering device per practical separating plate, or, in the case of structured packings, one metering device per structured packing. Preference is given to providing from 1 to 20, more preferably from 2 to 15, even more preferably from 5 to 15 and in particular from 7 to 13, metering devices in the upper section of the column for metering in an oxygen-containing gas.

In addition to the metering in according to the invention of the oxygen-containing gas in the upper section of the column, the same or another oxygen-containing gas may be metered in a manner known per se into the remaining section of the column, preferably into the bottom and more preferably into the bottom circuit.

Customarily, the mixture to be separated in the column is stabilized against polymerization using at least one stabilier. This at least one stabilier may be conducted into the column with the mixture and/or be introduced additionally into the column during the separation, for example using a recycle stream.

Examples of useful stabilizers include phenolic compounds, amines, nitro compounds, phosphorus or sulfur compounds, hydroxylamines, N-oxyls and certain inorganic salts, and also optionally mixtures thereof.

Preference is given to stabilizers such as phenothiazine, N-oxyls and phenolic compounds.

Examples of N-oxyls (nitroxyl or N-oxyl radicals, i.e. compounds containing at least one >N—O.group) include 4-hydroxy-2,2,6,6-tetramethylpiperidine N-oxyl, 4-oxo-2,2,6,6-tetramethylpiperidine N-oxyl, 4-acetoxy-2,2,6,6-tetramethylpiperidine N-oxyl, 2,2,6,6-tetramethylpiperidine N-oxyl or 3-oxo-2,2,5,5-tetramethylpyrrolidine N-oxyl.

Examples of phenolic compounds include alkylphenols, for example o-, m- or p-cresol (methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tertbutylphenol, 2-methyl-4-tert-butylphenol, 4-tert-butyl-2,6-dimethylphenol or 2,2′-methylenebis(6-tert-butyl-4-methylphenol), 4,4′-oxybiphenol, 3,4-methylenedioxyphenol (sesamol), 3,4-dimethylphenol, hydroquinone, catechol (1,2-dihydroxybenzene), 2-(1′-methylcyclohex-1′-yl)-4,6-dimethylphenol, 2- or 4-(1′-phenyleth-1′-yl)phenol, 2-tert.-butyl-6-methylphenol, 2,4,6-tris-tert-butylphenol, 2,6-di-tert-butylphenol, 2,4-di-tert-butylphenol, 4-tert-butylphenol, nonylphenol [11066-49-2], octylphenol [140-66-9], 2,6-dimethylphenol, bisphenol A, bisphenol F, bisphenol B, bisphenol C, bisphenol S, 3,3′,5,5′-tetrabromobisphenol A, 2,6-di-tert.-butyl-p-cresol, Koresin® from BASF AG, methyl 3,5-di-tert.-butyl-4-hydroxybenzoate, 4-tert.-butylcatechol, 2-hydroxybenzyl alcohol, 2-methoxy-4-methylphenol, 2,3,6-trimethylphenol, 2,4,5-trimethylphenol, 2,4,6-trimethylphenol, 2-isopropylphenol, 4-isopropylphenol, 6-isopropyl-m-cresol, n-octadecyl β-(3,5-di-tert.-butyl-4-hydroxyphenyl)propionate, 1,1,3-tris(2-methyl-4-hydroxy-5-tert-butylphenyl)butane, 1,3,5-trimethyl-2,4,6-tris(3,5-di-tert.-butyl-4-hydroxybenzyl)benzene, 1,3,5-tris(3,5-di-tert.-butyl-4-hydroxybenzyl) isocyanurate, 1,3,5-tris(3,5-di-tert.-butyl-4-hydroxyphenyl)propionyloxyethyl isocyanurate, 1,3,5-tris(2,6-dimethyl-3-hydroxy-4-tert-butylbenzyl) isocyanurate or pentaerythrityl tetrakis[β-(3,5-di-tert.-butyl-4-hydroxyphenyl)propionate], 2,6-di-tert-butyl-4-dimethylamino-methylphenol, 6-sec-butyl-2,4-dinitrophenol, Irganox® 565, 1141, 1192, 1222 and 1425 from Ciba Spezialitätenchemie, octadecyl 3-(3′,5′-di-tert-butyl-4′-hydroxyphenyl)propionate, hexadecyl 3-(3′,5′-di-tert-butyl-4′-hydroxyphenyl)propionate, octyl 3-(3′,5′-di-tert-butyl-4′-hydroxyphenyl)propionate, 3-thia-1,5-pentanediol bis[(3′,5′-di-tert-butyl-4′-hydroxyphenyl)propionate], 4,8-dioxa-1,11-undecanediol bis[(3″,5′-di-tert-butyl-4′-hydroxyphenyl)propionate], 4,8-dioxa-1,11-undecanediol bis[(3′-tert-butyl-4′-hydroxy-5′-methylphenyl)propionate], 1,9-nonanediol bis[(3′,5′-di-tert-butyl-4′-hydroxyphenyl)propionate], 1,7-heptanediamine-bis[3-(3′,5′-di-tert-butyl-4′-hydroxyphenyl)propionamide], 1,1-methanediamine-bis[3-(3′,5′-di-tert-butyl-4′-hydroxyphenyl)propionamide], 3-(3′,5′-di-tert-butyl-4′-hydroxyphenyl)propionic hydrazide, 3-(3′,5′-di-methyl-4′-hydroxyphenyl)propionic hydrazide, bis(3-tert-butyl-5-ethyl-2-hydroxyphen-1-yl)methane, bis(3,5-di-tert-butyl-4-hydroxyphen-1-yl)methane, bis[3-(1′-methylcyclohex-1′-yl)-5-methyl-2-hydroxyphen-1-yl]methane, bis(3-tert-butyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl) sulfide, bis(3-tert-butyl-2-hydroxy-5-methylphen-1-yl) sulfide, 1,1-bis(3,4-dimethyl-2-hydroxyphen-1-yl)-2-methylpropane, 1,1-bis(5-tert-butyl-3-methyl-2-hydroxyphen-1-yl)butane, 1,3,5-tris[1′-(3″,5′-di-tert-butyl-4″-hydroxyphen-1″-yl)meth-1′-yl]-2,4,6-trimethylbenzene, 1,1,4-tris(5′-tert-butyl-4′-hydroxy-2‘-methylphen-l’-yl)butane, aminophenols such as para-aminophenol, nitrosophenols such as para-nitrosophenol, p-nitroso-o-cresol, alkoxyphenols, for example 2-methoxyphenol (guajacol, catechol monomethyl ether), 2-ethoxyphenol, 2-isopropoxyphenol, 4-methoxyphenol (hydroquinone monomethyl ether), mono- or di-tert-butyl-4-methoxyphenol, 3,5-di-tert-butyl-4-hydroxyanisole, 3-hydroxy-4-methoxybenzyl alcohol, 2,5-dimethoxy-4-hydroxybenzyl alcohol (syringa alcohol), 4-hydroxy-3-methoxybenzaldehyde (vanillin), 4-hydroxy-3-ethoxybenzaldehyde (ethylvanillin), 3-hydroxy-4-methoxybenzaldehyde (isovanillin), 1-(4-hydroxy-3-methoxyphenyl)ethanone (acetovanillone), eugenol, dihydroeugenol, isoeugenol, tocopherols such as α-, β-, γ-, δ- and ε-tocopherol, tocol, α-tocopherolhydroquinone and also 2,3-dihydro-2,2-dimethyl-7-hydroxybenzofuran (2,2-dimethyl-7-hydroxycoumaran), quinones and hydroquinones such as hydroquinone, 2,5-di-tert-butylhydroquinone, 2-methyl-p-hydroquinone, 2,3-dimethylhydroquinone, trimethylhydroquinone, 4-methylcatechol, tert.-butylhydroquinone, 3-methylcatechol, benzoquinone, 2-methyl-p-hydroquinone, 2,3-dimethylhydroquinone, trimethylhydroquinone, 3-methylcatechol, 4-methylcatechol, tert.-butylhydroquinone, 4-ethoxyphenol, 4-butoxyphenol, hydroquinone monobenzyl ether, p-phenoxyphenol, 2-methylhydroquinone, 2,5-di-tert-butylhydroquinone, tetramethyl-p-benzoquinone, diethyl 1,4-cyclohexanedione-2,5-dicarboxylate, phenyl-p-benzoquinone, 2,5-dimethyl-3-benzyl-p-benzoquinone, 2-isopropyl-5-methyl-p-benzoquinone (thymoquinone), 2,6-diisopropyl-p-benzoquinone, 2,5-dimethyl-3-hydroxy-p-benzoquinone, 2,5-dihydroxy-p-benzoquinone, embelin, tetrahydroxy-p-benzoquinone, 2,5-dimethoxy-1,4-benzoquinone, 2-amino-5-methyl-p-benzoquinone, 2,5-bisphenylamino-1,4-benzoquinone, 5,8-dihydroxy-1,4-naphthoquinone, 2-anilino-1,4-naphthoquinone, anthraquinone, N,N-dimethylindoaniline, N,N-diphenyl-p-benzoquinone diimine, 1,4-benzoquinone dioxime, coerulignone, 3,3′-di-tert.-butyl-5,5′-dimethyldiphenoquinone, p-rosolic acid (aurin), 2,6-di-tert.-butyl-4-benzylidenebenzoquinone or 2,5-di-tert-amylhydroquinone.

Aromatic amines are, for example, N,N-diphenylamine; phenylenediamines are, for example, N,N′-dialkyl-para-phenylenediamine, where the alkyl radicals may each independently contain from 1 to 4 carbon atoms and may be linear or branched, for example, N,N′-di-sec.-butyl-para-phenylenediamine; hydroxylamines are, for example, N,N-diethylhydroxylamine; phosphorus compounds are, for example, triphenylphosphine, triphenyl phosphite or triethyl phosphite, sulfur compounds are, for example, diphenyl sulfide and inorganic salts are, for example, the chloride, dithiocarbamate, sulfate, salicylate and acetate salts of copper, manganese, cerium, nickel and chromium.

Preference is given to phenothiazine, p-aminophenol, p-nitrosophenol, 2-tert-butylphenol, 4-tert-butylphenol, 2,4-di-tert-butylphenol, 2-methyl-4-tert-butylphenol, 4-tert-butyl-2,6-dimethylphenol, hydroquinone and/or hydroquinone monomethyl ether, N,N′-di-sec.-butyl-para-phenylenediamine and also manganese(II) acetate, cerium(III) carbonate or cerium(III) acetate; particular preference is given to phenothiazine, p-aminophenol, p-nitrosophenol, 2-tert-butylphenol, 4-tert-butylphenol, 2,4-di-tert-butylphenol, 2-methyl-4-tert-butylphenol, 4-tert-butyl-2,6-dimethylphenol, hydroquinone and/or hydroquinone monomethyl ether, cerium(III) acetate and/or manganese(II) acetate.

Very particular preference is given to phenothiazine, hydroquinone monomethyl ether, manganese(II) acetate and a mixture of hydroquinone monomethyl ether and phenothiazine or phenothiazine, hydroquinone monomethyl ether and manganese(II) acetate.

The way in which the stabilizer is added is not limited. The stabilizer added may in each case be added individually or as a mixture, in liquid form or in dissolved form in a suitable solvent which may itself be a stabilizer, as described, for example, in the previous German patent application having the reference number 102 00 583.4.

The stabilizer may, for example, be added in a suitable formulation at any desired point in the column, to an external cooling circuit or to a suitable recycle stream. Preference is given to adding directly into the column or into a recycle stream.

When a mixture of a plurality of stabilizers is used, these may be fed independently at different metering points or at the same metering point as mentioned above.

When a mixture of a plurality of stabilizers is used, these may also be dissolved independently in different solvents.

Depending on the individual substance, the concentration of the stabilizer in the column may be between 1 and 10 000 ppm, preferably between 10 and 5000 ppm, more preferably between 30 and 2500 ppm and in particular between 50 and 1500 ppm.

Particular preference is given to spraying the dissolved stabilizer (mixture) onto any condenser surfaces, column internals or column lids present.

The product removed from the column, i.e. (meth)acrylic acid or (meth)acrylic ester, may have any desired purities which are not essential to the invention, for example at least 90%, preferably at least 95%, more preferably at least 98% and most preferably at least 99%.

In the case of crude acrylic acid which may, for example, be removed in a sidestream takeoff, the following may generally be present in addition to acrylic acid:

| | |
|---|---|
| from 0.1 to 2% | of lower carboxylic acids, for |
| by weight | example acetic acid, propionic acid |
| from 0.5 to 5% | of water |
| by weight | |
| from 0.05 to 1% | of low molecular weight aldehydes, |
| by weight | e.g. benzaldehyde, 2- or 3-furfural, |
| | acrolein |
| from 0.01 to 1% | of maleic acid and/or its anhydrides |
| by weight | |
| from 1 to 500 ppm | of stabilizer, |
| by weight | |

based in each case on the weight of the crude acrylic acid.

Glacial acrylic acid purified in the column may, for example, have the following composition:

| | |
|---|---|
| acrylic acid | 99.7-99.9% by |
| | weight |
| acetic acid | 50-1500 ppm by |
| | weight |
| propionic acid | 10-500 ppm by |
| | weight |
| diacrylic acid | 10-1000 ppm by |
| | weight |
| water | 50-1000 ppm by |
| | weight |
| aldehyde and other | 1-50 ppm by |
| carbonylics | weight |
| inhibitors | 100-300 ppm by |
| | weight |
| maleic acid/anhydride | 1-20 ppm by |
| | weight |

The process according to the invention for working up (meth)acrylic acid or (meth)acrylic esters is preferably part of an overall process for preparing (meth)acrylic acid or (meth)acrylic esters which, in a preferred embodiment for acrylic acid, comprises the following steps:

(a) catalytic gas phase oxidation of propane, propene and/or acrolein to acrylic acid to obtain a gaseous reaction product containing acrylic acid,

(b) absorption of the reaction product with a solvent,

(c) distillation of the solvent laden with reaction product to obtain crude acrylic acid and the solvent,

(d) optionally purifying the crude acrylic acid by crystallization and

(e) optionally esterifying the crude or crystallized acrylic acid.

Stage a

According to the invention, the C₃ starting compounds may be catalytically reacted in the gas phase with molecular oxygen to give acrylic acid by known processes as described above.

The conversion of propene to acrylic acid is strongly exothermic. The reaction gas which, in addition to the reactants and products, advantageously comprises a diluent gas, for example cycle gas, nitrogen from air and/or steam, can therefore only take up a small portion of the heat of reaction. Although the type of reactors used is subject to no restriction per se, tube bundle heat exchangers are usually used which are charged with oxidation catalyst, since they are capable of removing the predominant portion of the heat released in the reaction by convection and radiation at the cooled tube walls.

However, stage (a) does not provide pure acrylic acid, but rather a gaseous mixture which, in addition to acrylic acid, comprises secondary components which are substantially unconverted acrolein and/or propene, steam, carbon monoxide, carbon dioxide, nitrogen, oxygen, acetic acid, propionic acid, formaldehyde, further aldehydes and maleic anhydride.

Typically, the reaction product mixture, based in each case on the entire reaction mixture, comprises from 0.05 to 1% by weight of propene and from 0.05 to 1% by weight of acrolein, from 0.01 to 2% by weight of propane, from 1 to 20% by weight of steam, from 0.05 to 15% by weight of carbon oxides, from 10 to 90% by weight of nitrogen, from 0.05 to 5% by weight of oxygen, from 0.05 to 2% by weight of acetic acid, from 0.01 to 2% by weight of propionic acid, from 0.05 to 1% by weight of formaldehyde, from 0.05 to 2% by weight of aldehydes and also from 0.01 to 0.5% by weight of maleic anhydride.

Stage b

In stage (b), the acrylic acid and a portion of the secondary components from the reaction gas is removed by absorption with a solvent. According to the invention, useful solvents are water or especially all high-boiling solvents, preferably solvents having a boiling point above 160° C. A particularly suitable solvent is a mixture of diphenyl ether and biphenyl, especially the commercially obtainable mixture of 75% by weight of diphenyl ether and 25% by weight of biphenyl, to which, as mentioned above, ortho-phthalic ester may be added.

In the present context, the terms high boilers, medium boilers and low boilers and also corresponding terms used as adjectives refer to compounds which have a higher boiling point than acrylic acid (high boilers), those which have approximately the same boiling point as acrylic acid (medium boilers), and those which have a lower boiling point than acrylic acid (low boilers).

Advantageously, the hot reaction gas obtained from stage (a) is cooled before the absorption by partially evaporating the solvent in a suitable apparatus, for example a direct condenser or quench apparatus. Useful apparatus for this purpose include Venturi scrubbers, bubble columns and spray condensers.

The high-boiling secondary components of the reaction gas from stage (a) condense into the unevaporated solvent. The partial evaporation of the solvent is also a purification step for the solvent. In a preferred embodiment of the invention, a substream of the unevaporated solvent, preferably from 1 to 10% of the mass stream fed to the absorption column is removed and subjected to solvent purification. In this purification, the solvent is distilled over and the high-boiling secondary components which remain behind may be disposed of, for example incinerated, if required in more highly concentrated form. This solvent distillation serves to avoid too high a concentration of high boilers in the solvent stream.

The absorption is effected in a countercurrent absorption column which is preferably equipped with valve and/or dual-flow trays and is charged from above with (unevaporated) solvent. The gaseous reaction product and any evaporated solvent are passed from below into the column and then cooled to absorption temperature. The cooling is advantageously effected by cooling circuits, i.e. preheated solvent is removed from the column, cooled in heat exchangers and fed back to the column at a point above the takeoff point. In addition to acrylic acid, these solvent cooling circuits also condense low-, high- and medium-boiling secondary components and also evaporated solvent. As soon as the reaction gas stream is cooled to the absorption temperature, the actual absorption is effected. The remainder of the acrylic acid in the reaction gas is absorbed, as is a portion of the low-boiling secondary components.

The remaining, unabsorbed reaction gas from stage (a) is cooled further, in order to remove the condensable portion of the low-boiling secondary components thereof, in particular water, formaldehyde and acetic acid, by condensation. This condensate is referred to hereinbelow as acid water. The remaining gas stream, referred to hereinbelow as cycle gas, consists predominantly of nitrogen, carbon oxides and unconverted reactants. Preference is given to feeding some of this gas stream back to the reaction stages as diluent gas.

A solvent stream laden with acrylic acid, high- and medium-boiling secondary components and also a small portion of low-boiling secondary components is removed from the bottom of the column used in stage (b) and, in a preferred configuration of the invention, subjected to a desorption. This is advantageously carried out in a column which may preferably be equipped with valve and/or dual-flow trays but also with random packings or structured packings, in the presence of what is known as a stripping gas. The stripping gas used may be any inert gas or gas mixture, although preference is given to using a gas mixture of air and nitrogen or cycle gas, since this occurs in stage (a) when carrying out an evaporation of a portion of the solvent. The desorption strips the majority of the low boilers out of the laden solvent using a portion of the cycle gas which is removed before stage (a). Since relatively large amounts of acrylic acid are also stripped, this stream, referred to hereinbelow as stripping cycle gas, is for economic reasons advantageously not discarded, but instead recirculated, for example to the stage at which the solvent is partially evaporated or into the absorption column. Since the stripping gas is part of the cycle gas, it still contains significant amounts of low boilers itself. The performance of the column used for desorption can be increased when the low boilers are removed from the stripping gas before it is passed into the column. In terms of process engineering, this is advantageously carried out in such a way that the stripping gas is purified using solvent worked up in stage (c) described below in a countercurrent scrubbing column.

A virtually low boiler-free, acrylic acid-laden solvent stream may then be removed from the bottom of the column used for the desorption.

Stage c

In process stage (c), the acrylic acids together with the medium-boiling components and also the last residue of low-boiling secondary components are removed from the solvent. This separation is effected by means of distillation, in principle using any distillation column. Preference is given to using a column having sieve trays, for example dual-flow trays, valve trays or crossflow sieve trays made of metal. In the rectifying section of the column, the acrylic acid is distilled to free it of solvent and the medium-boiling secondary components such as maleic anhydride. In order to reduce the proportion of low boilers in the acrylic acid, the rectifying section of the column is advantageously lengthened and the acrylic acid is removed from the column as a sidestream takeoff. This acrylic acid is-referred to hereinbelow, irrespective of its purity, as crude acrylic acid.

At the top of the column, a low boiler-rich stream is then removed after a partial condensation. However, since this stream still contains acrylic acid, it is advantageously not discarded, but instead recycled to the absorption stage (b).

The low boiler-free and virtually acrylic acid-free solvent is removed from the bottom of the column, and the majority thereof is preferably fed to the countercurrent scrubbing column in which the stripping gas of stage (b) is purified, in order to scrub the low boilers out of the stripping gas. The virtually acrylic acid-free solvent is then fed to the absorption column.

According to the invention, this column, as described above, is provided with at least one means of metering oxygen-containing gases in the upper section of the column.

It will be appreciated that all apparatus, especially the columns in which an acrylic acid-containing stream is purified, are operated in the manner according to the invention by metering in an oxygen-containing gas in its upper section.

In a preferred embodiment of the invention, the dilute aqueous acid which may still contain dissolved acrylic acid is treated extractively with a small substream of the virtually acrylic acid-free solvent. This acid water extraction extracts a portion of the acrylic acid in the solvent and thus recovers it from the acid water. At the same time, the acid water extracts the polar medium-boiling components from the solvent stream and thus avoids an increase in these components in the solvent circuit. The acid water stream composed of low and middle boilers may be further concentrated.

The crude acrylic acid obtained in stage (c), based in each case on the crude acrylic acid, preferably comprises from 98 to 99.8% by weight, in particular from 98.5 to 99.5% by weight, of acrylic acid, and from 0.2 to 2% by weight, in particular from 0.5 to 1.5% by weight, of impurities, for example acetic acid, aldehydes and maleic anhydride. This acrylic acid may, as long as the requirements of its purity are not too high, optionally be used directly for esterification.

Stage (d):

In stage (d), the crude acrylic acid obtained in step (c) may be further purified by distillation or crystallization, preferably by means of fractional crystallization by a combination of dynamic and static crystallization. The type of distillation or crystallization here is subject to no particular restriction.

In the static crystallization (for example U.S. Pat. No. 3,597,164 and FR 2 668 946), the liquid phase is moved only by free convection, while in the dynamic crystallization, the liquid phase is moved by forced convection. The latter may be effected by forced flow in apparatus having full flow-through (cf. DE 26 06 364) or by applying a sprayed film or falling film to a cooled wall (DT 1 769 123 and EP 218 545).

Stage (e):

If desired, the crude or glacial acrylic acid obtained in stage (c) or (d) may be esterified by known methods.

To this end, the esterification methods known from the prior art may be used, for example as described in the German patent application having the reference number 101 44 490.7, EP-A 733 617, EP-A 1 081 125, DE-A 196 04 267 or DE-A 196 04 253. In the course of the preparation and/or workup of the ester, an oxygen-containing gas may be metered in in accordance with the invention in the upper section of one or more columns, preferably in the column in which the ester is purified by distillation.

The present invention makes it possible to operate columns for distillative, rectificative or fractional workup of (meth)acrylic acid or (meth)acrylic esters within an optimum range in which, on the one hand, the inhibitor used is supported by the costabilizing effect of oxygen and, on the other hand, the gas ballast of the column is reduced and a relatively low output is therefore sufficient for the connected vacuum unit.

ppm and percentage data used in this document refer, unless otherwise stated, to percentages by weight and ppm by weight.

### EXAMPLES

### Example 1

In a 500 ml four-neck flask equipped with a jacketed coil condenser, gas inlet tube and thermoelements, 300 g of 99.9% acrylic acid were initially charged. The acrylic acid used was stabilized with 275 ppm of phenothiazine. In addition, a gas mixture of 4.8 l/h of nitrogen and 30 ml/h of air was passed through the initially charged acrylic acid. The initially charged acrylic acid was maintained at a temperature of 25° C. for 16 hours, then the four-neck flask was immersed in a heating bath preheated to 80° C.

The partial oxygen pressure above the initially charged acrylic acid was 1.2 mbar. The oxygen content (pₒ₂) above the liquid was 0.12% by volume. After 22 hours, the initially charged acrylic acid became cloudy owing to precipitated polymer.

### Examples 2 to 8

The procedure was as described under example 1. 99.7% acrylic acid was used. The acrylic acid used was stabilized with 286 ppm of phenothiazine.

| | | | | | Clouding | | |
|---|---|---|---|---|---|---|---|
| Example | Nitrogen | Air | Po2 | | after | | O₂ content |
| 2 | — | 4.00 1/h | 190 | mbar | >214 | h | 19.0% by |
| | | | | | | | volume |
| 3 | 4.0 1/h | 0.10 1/h | 4.6 | mbar | >264 | h | 0.46% by |
| | | | | | | | volume |
| 4 | 4.0 1/h | 0.05 1/h | 2.3 | mbar | >314 | h | 0.23% by |
| | | | | | | | volume |
| 5 | 6.25 1/h | 0.05 1/h | 1.5 | mbar | 21 | h | 0.15% by |
| | | | | | | | volume |
| 6 | 4.8 1/h | 0.03 1/h | 1.2 | mbar | 1.5 | h | 0.12% by |
| | | | | | | | volume |
| 7 | 10.0 1/h | 0.03 1/h | 0.5 | mbar | 1 | h | 0.05% by |
| | | | | | | | volume |
| 8 | 4.0 1/h | — | 0.0 | mbar | 0.5 | h | — |

In examples 5 to 7, the four-neck flask contained thin polymer deposits after 24 hours; the initially charged acrylic acid was still mobile.

In example 8, the initially charged acrylic acid was polymerized through after 15 hours.

### Example 9

The procedure was as under example 1. 99.7% acrylic acid was used. The acrylic acid used had been stabilized with 283 ppm of phenothiazine, 100 ppm of 4-hydroxy-2,2,6,6-tetramethylpiperidine N-oxyl and 80 ppm of hydroquinone monomethyl ether. In addition, a gas mixture of 10 l/h of nitrogen and 30 ml/h of air was passed through the initially charged acrylic acid.

The partial oxygen pressure above the initially charged acrylic acid was 0.5 mbar. The oxygen content above the liquid was 0.05% by volume. After 2 hours, the initially charged acrylic acid became cloudy owing to precipitated polymer.

### Examples 10 to 13

The procedure was as under example 1. 99.7% acrylic acid was used. The acrylic acid used had been stabilized with 284 ppm of hydroquinone monomethyl ether and 12.5 ppm of manganese(II) acetate tetrahydrate.

| | | | | | Clouding | | |
|---|---|---|---|---|---|---|---|
| Example | Nitrogen | Air | Po2 | | after | | O₂ content |
| 10 | — | 4.00 1/h | 190 | mbar | >184 | h | 19.0% by |
| | | | | | | | volume |
| 11 | 4.0 1/h | 0.05 1/h | 2.3 | mbar | >178 | h | 0.23% by |
| | | | | | | | volume |
| 12 | 10.0 1/h | 0.03 1/h | 0.5 | mbar | 5 | h | 0.05% by |
| | | | | | | | volume |
| 13 | 4.0 1/h | — | 0.0 | mbar | 1 | h | — |

### Examples 14 to 16

The procedure was as under example 1. 99.7% acrylic acid was used. The acrylic acid used had been stabilized with 286 ppm of phenothiazine. The runs were carried out at a pressure of 500 bar.

| | | | | Clouding | | |
|---|---|---|---|---|---|---|
| Example | Nitrogen | Air | Po2 | after | | O₂ content |
| 14 | — | 4.00 1/h | 83 mbar | >314 | h | 16.6% by |
| | | | | | | volume |
| 15 | 4.0 1/h | 0.10 1/h | 2.1 mbar | >158 | h | 0.41% by |
| | | | | | | volume |
| 16 | 4.0 1/h | 0.05 1/h | 1.0 mbar | 1.5 | h | 0.21% by |
| | | | | | | volume |

We claim:

1. A process for working up mixtures comprising (meth)acrylic acid and/or (meth)acrylic ester in a column for distilling, rectifying and/or fractionally condensing in the presence of at least one polymerization inhibitor and an oxygen-containing gas, wherein the partial oxygen pressure p(O₂) in the gas phase of the entire column is from 2 to 5 hPa.

2. A process as claimed in claim 1, wherein the process is carried out in the presence of at least one anaerobic polymerization inhibitor.

3. A process as claimed in claim 2, wherein the anaerobic polymerization inhibitor is phenothiazine.

4. A process as claimed in any of the preceding claims, wherein the process is carried out in the presence of at least one polymerization inhibitor selected from the group of phenothiazine, p-aminophenol, p-nitrosophenol, 2-tert-butylphenol, 4-tert-butylphenol, 2,4-di-tert-butylphenol, 2-methyl-4-tert-butylphenol, 4-tert-butyl-2,6-dimethylphenol, hydroquinone, hydroquinone monomethyl ether, N,N′-di-sec-butyl-para-phenylenediamine, manganese(II) acetate, cerium(III) carbonate and cerium(III) acetate.

5. A process as claimed in any of the preceding claims, wherein the process is carried out in the presence of phenothiazine, hydroquinone monomethyl ether, manganese(II) acetate, a mixture of hydroquinone monomethyl ether and phenothiazine or a mixture of phenothiazine, hydroquinone monomethyl ether and manganese(II) acetate.
